(19) United States
(12) Patent Application Publication
(10) Pub. No.: US 2004/0260269 A1
(43) Pub. Date: Dec. 23, 2004

(54) Method for treating false aneurysm and a needle for use in such a method

(76) Inventor: Yair Assaf, Modiin (IL)

Correspondence Address: NATH & ASSOCIATES PLLC, Sixth Floor, 1030 15th Street, N.W., Washington, DC, 20005, US

(21) Appl. No.: 10/601,312

(22) Filed: Jun. 23, 2003

(51) Int. Cl. A61M 31/00

(52) U.S. Cl. 604/506; 427/2.1; 600/431; 604/264

(57) ABSTRACT

A method for improving the ultrasonic visibility of medical devices is provided. The method comprises coating, at least part of the device, with a heavy metal, i.e. a metal having a density of at least 12 g/cc. Preferable metals are biologically inert ones, such as gold, rhodium, and platinum. Also provided by the present invention is an injection needle, the ultrasonic visibility of which is increased by coating at least a portion thereof with a heavy metal. Finally, a method for treating false aneurysm is provided, by ultrasound guided thrombin injection, wherein the injection is carried out with a needle according to the invention.

### FIELD OF THE INVENTION

This invention relates to a method for treating false aneurysm and to a needle for use in such a method.

### BACKGROUND OF THE INVENTION

When a subject undergoes catheterization, it happens, in about 1% of the cases, that an artery is inadvertently punctured, and blood flows out therefrom, clotting in the vicinity thereof. This condition is named pseudoaneurysm or false aneurysm (cFA).

cFA was treated surgically until 1991, when Ultrasound Guided Compression-closure (UGC) was introduced. UGC rapidly replaced surgery as the treatment modality of first choice. It still remains the most popular treatment, although another solution was suggested already about 6 years ago, in 1997, when Liau et al. suggested Ultrasound Guided Thrombin Injection (UGTI) to treat false aneurysm.

In UGTI, the physician injects thrombin to the center of the aneurysm, but outside the affected artery, such that the artery is not punctured by the injecting needle, and thrombin is not injected into the artery, an incident that might result in the artery's occlusion. To locate the exact position of the injection needle, Ultrasound imaging is used.

### RELATED ART

Some references that may be relevant to the understanding of the UGTI method or that may be relevant more generally as background to the present invention are listed below. Appearance of a document in this list should not be construed as implying that the document is relevant to the patentability of the claimed invention.

1. Paulson et al. (Radiology, 215 (2000) pages 403-408 “Treatment of latrogenic Femoral Pseudoaneurysms: Comparison of US guided Thrombin Injection with Compression Repair”;

2. Khoury et al., J. Vsc. Surg. 35(3) (2002) pp. 517-521 “Duplex scanning-guided thrombin injection for the treatment of iatrogenic pseudoaneurysms”;

3. Weinman et al., Eur. Vasc. Endovasc. Durg. 23, (2002) pp. 68-72 “Treatment of Postcatheterisation False Aneurysms: Ultrasound-guided Compression vs Ultrasound Guided Thrombin Injection;

4. WO 00/51136 “Medical tools and devices with improved ultrasound visibility”;

5. U.S. Pat. No. 5,081,997 “Echogenic devices, material and method”.

### SUMMARY OF THE INVENTION

According to one aspect of the present invention there is provided a method for increasing the ultrasonic visibility of a therapeutic device, comprising coating at least part of said device with an echogenic material, said echogenic material being a heavy metal.

In the context of the present description and claims, a heavy metal is a metal or a metal alloy having a density of more than 12 g/cc, preferably more than 15 g/cc Non-limiting examples of heavy metals are gold, platinum, rhodium, tantalum, rhenium, tungsten, osmium, iridium, and alloys thereof. Preferably, the heavy metal used in accordance with the invention is a biologically inert metal, such as gold, platinum, or rhodium, the insertion thereof into a patient's body is allowed, but it may also be a metal of another kind, provided that said metal coating is further coated with a biologically inert material.

Preferably, the part of the surface that is coated with a metal is at least 3 mm long around the entire circumference of the therapeutic device. Although it is possible to coat the device in its entirety, there is usually no need to coat more than lcm thereof, in order to obtain the desired ultrasonic visibility.

Ultrasonic visibility of an article should be construed as the visibility of said article during ultrasound imaging.

Ultrasound imaging is carried out by ultrasound waves transmitted thereon from an ultrasound source which is independent of said article.

Therapeutic device is any device that is intended for use in a therapeutic procedure, particularly such devices that are to be inserted into a subject and monitored by ultrasound imaging to allow precise determination of their position within the subject's body.

The heavy metal coating is at least about 5 μm thick, preferably about 10 μm thick. Experiments with needles coated with gold of 15 and 20 μm thickness are currently underway.

According to one embodiment of the present invention the therapeutic device is an injection needle, for example, of the kind used for spinal anesthesia, or a 19-22 gauge needle.

Also provided by the present invention is an injection needle coated by the method of the invention.

A needle according to the invention is typically between about 3 and about 12 cm long, preferably between around 9 and 10 cm long.

According to another aspect of the present invention there is provided a method for treating a subject having a false aneurysm affecting a blood vessel thereof, comprising injecting to said subject a blood-clotting agent, such as thrombin, into the false aneurysm, outside the affected blood vessel, wherein said agent is injected via an injection needle, which is coated with an echogenic material.

### BRIEF DESCRIPTION OF THE DRAWINGS

In order to understand the invention and to see how it may be carried out in practice, specific embodiments will now be described, by way of non-limiting examples only, with reference to the accompanying drawings, in which:

FIG. 1 is an illustration of an injection needle according to the invention;

FIG. 2 is an ultrasound image of a false aneurysm with two needles: one according to the invention, and the other a conventional needle.

### DETAILED DESCRIPTION OF THE DRAWINGS

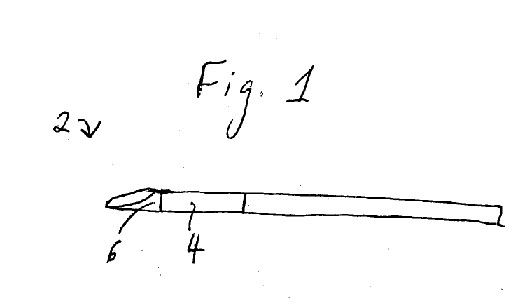

FIG. 1 shows an injection needle 2, being a therapeutic device the ultrasonic visibility of which is increased by coating its part 4 with gold.

The coating of the needle 2 is made of gold alloy of the type known in the art as hard gold, such as defined under Mil G 45204 standard.

The gold coating of needle 2 is 10 μm thick, to allow enhanced visibility in the ultrasound imaging. The tip of the needle 6 is not coated, since coating thereof may result in a less sharp needle-tip, and may cause unnecessary pain when inserted into the subject body. The length of the non-coated tip portion 6 of the needle 2 is about 2 mm.

Therapeutic devices may be coated with metals having densities of more than 12 g/cc in order to improve their ultrasonic visibility by any means known in the art per se.

The injection needle of FIG. 1 was first cleaned in a hot soak to remove oily residues that may have been present thereon. Then, it was activated by a 30% HCl solution in water. A thin layer of approximately 0.2 μm of Nickel was applied to the activated surface of the needle, and the needle was finally coated with gold by insertion into a commercial bath (Lea-Ronal, manufactured by Enthone, USA) for coating with hard gold type 3, to produce a coating with hardness of about 150-200 Knoop. Hard gold type 4 may also be useful.

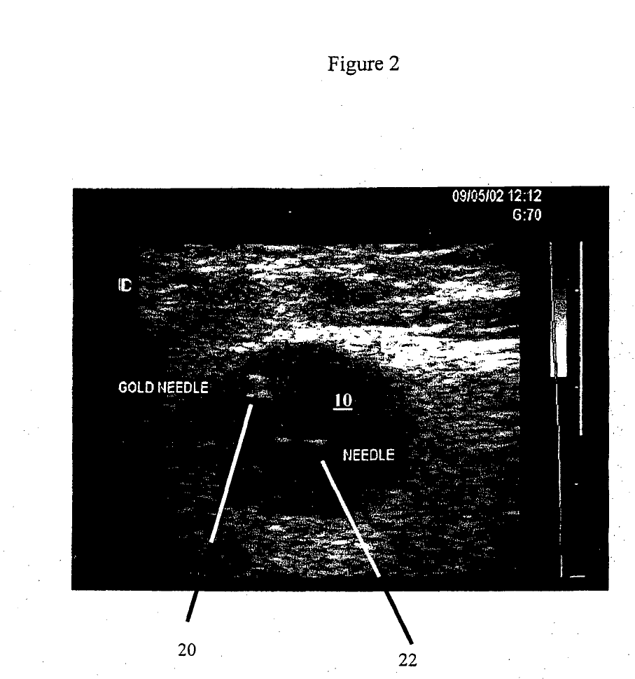

FIG. 2 is an ultrasound image taken from a subject having a false aneurysm 10 affecting a blood vessel (not shown). The Figure shows needles 20 and 22 through which thrombin is to be injected into the aneurysm 10. The needle 20 is of the kind described in FIG. 1, i.e. coated with 10 μm gold, the needle 22 is a conventional needle made of stainless-steal, without an echogenic coating. It is clear from the picture that the needle 20 has a much better visibility than the needle 22.

FIG. 2 was taken during treatment with UGTI method. This method is fully described in ref. 1-3 above. Naturally, the therapeutic device of the invention may also be used in other procedures that require enhanced ultrasonic visibility, such as certain kinds of biopsy, gynecologic procedures, and the like.

1. A method for increasing the ultrasonic visibility of a therapeutic device, comprising coating at least part of said device with a metal having density of more than 12 g/cc.

2. A method according to claim 1, wherein said density is more than 15 g/cc.

3. A method according to claim 1, wherein said metal is selected from the group consisting of gold, platinum, rhodium, tantalum, rhenium, tungsten, osmium, iridium, and alloys thereof.

4. A method according to claim 1 wherein said metal is biologically inert, such that its insertion into a patient's body is allowed.

5. A method according to claim 4, wherein said metal is selected from the group consisting of gold, platinum, rhodium, and alloys thereof.

6. A method according to claim 5 wherein said metal is gold or alloys thereof.

7. A method according to claim 6 wherein said gold alloy is defined under Mil G 45204 standard.

8. A method according to claim 1 wherein said metal is coated with a biologically inert coating.

9. A method according to claim 8 wherein said metal is not biologically inert.

10. A method according to claim 1 wherein said therapeutic device is insertable into the body of a subject.

11. A method according to claim 1 wherein said therapeutic device is an injection needle.

12. A method according to claim 1 wherein said coating is at least about 5 μm thick.

13. A method according to claim 6 wherein said coating is about 10 μm thick.

14. A method for treating a subject having a false aneurysm affecting a blood vessel thereof, comprising injecting into said false aneurysm, outside said affected blood vessel, a blood-clotting agent, wherein said blood-clotting agent is injected via an injection needle, which is at least partially coated with an echogenic material.

15. A method according to claim 14 wherein said echogenic material is a metal having density of more than 12 g/cc.

16. A method according to claim 15 wherein said density is more than 15 g/cc.

17. A method according to claim 15 wherein said metal is selected from the group consisting of gold, platinum, rhodium, tantalum, rhenium, tungsten, osmium, iridium, and alloys thereof.

18. A method according to claim 15 wherein said metal is biologically inert, such that its insertion into a patient's body is allowed.

19. A method according to claim 18 wherein said metal is selected from gold, platinum, rhodium, and alloys thereof.

20. A method according to claim 19 wherein said metal is gold or alloys thereof.

21. A method according to claim 20 wherein said gold alloy is defined under Mil G 45204 standard.

22. A method according to claim 14 wherein said echogenic material is coated with a biologically inert material.

23. An injection needle having a tip, said needle being at least partially coated with a metal having a density of more than 12 g/cc.

24. An injection needle according to claim 23 wherein said density is more than 15 g/cc.

25. An injection needle according to claim 23 wherein said metal is selected from gold, platinum, rhodium, tantalum, rhenium, tungsten, osmium, iridium, and alloys thereof.

26. An injection needle according to claim 25 wherein said metal is biologically inert, such that its insertion into a patient's body is allowed.

27. An injection needle according to claim 26, wherein said metal is selected from gold, platinum, rhodium and alloys thereof.

28. An injection needle according to claim 27 wherein said metal is gold or an alloy thereof.

29. An injection needle according to claim 28 wherein said gold alloy is defined under Mil G 45204 standard.

30. An injection needle according to claim 23, wherein said metal is further coated with a biologically inert material.

31. An injection needle according to claim 23, suitable for use for spinal anesthesia.

32. An injection needle according to claim 23, having a length between about 3 and about 12 cm.

33. An injection needle according to claim 32, wherein said length is about 10 cm.

34. An injection needle according to claim 23, having a 20-22 gauge.

35. An injection needle according to claim 23, wherein said tip is not coated with said echogenic material.

36. An injection needle according to claim 35, wherein the length of the tip that is not coated by the echogenic material is about 2 mm.

37. An injection needle according to claim 23, wherein said echogenic material has thickness of at least about Spm.

38. An injection needle according to claim 37, wherein said thickness is about 10 μm.
